(19) United States
(12) Patent Application Publication
(10) Pub. No.: US 2009/0131815 A1
(43) Pub. Date: May 21, 2009

(54) MARKER DEPLOYMENT DEVICE

(76) Inventors: Brian Zimmer, Indianapolis, IN (US); Michael Hoffa, Brownsburg, IN (US)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 11/943,304

(22) Filed: Nov. 20, 2007

(51) Int. Cl. A61B 10/00

(52) U.S. Cl. 600/562

(57) ABSTRACT

A marker deployment system is provided, including at least one cannula, a site marker and at least one wire. The cannula has a tubular side-wall and a distal opening. The cannula defines at least one aperture extending through the side-wall. The wire is selectively interposed within the cannula, and a distal wire end extends through the aperture. A portion of the wire is secured to an end portion of the site marker. The wire is pulled taut at the aperture, thereby at least partially aligning the end portion of the site marker with the distal opening of the cannula.

### TECHNICAL FIELD

The present disclosure relates generally to marker deployment devices for biopsy procedures. More specifically, the present disclosure relates to a marker deployment system including a cannula, a site marker and at least one wire selectively interposed within the cannula.

### BACKGROUND

In the diagnosis and treatment of breast cancer, it is often necessary to perform a biopsy to remove tissue samples from a suspicious mass. The suspicious mass is typically discovered during a preliminary examination involving visual examination, palpation, X-ray, magnetic resonance imaging (MRI), ultrasound imaging or other detection means.

When a suspicious mass is detected, a sample is taken by biopsy, and then tested to determine whether the mass is malignant or benign. This biopsy procedure can be performed by an open surgical technique, or through the use of a specialized biopsy instrument. To minimize surgical intrusion, a small specialized instrument such as a biopsy needle is inserted in the breast while the position of the needle is monitored using fluoroscopy, ultrasonic imaging, X-rays, MRI or other suitable imaging techniques.

Regardless of the method or instrument used to perform the biopsy, subsequent examination of the surgical site may be necessary, either in a follow up examination or for treatment of a cancerous lesion. Treatment often includes a mastectomy, lumpectomy, radiation therapy, or chemotherapy procedure that requires the surgeon or radiologist to direct surgical or radiation treatment to the precise location of the lesion. Because this treatment might extend over days or weeks after the biopsy procedure, and the original features of the tissue may have been removed or altered by the biopsy, it is desirable to insert a site marker into the surgical cavity to serve as a landmark for future identification of the location of the lesion.

However, some biopsy site markers may not be visible under all available modalities. When cancer is found at a biopsy site that has been previously marked with a site marker, the poor visibility of the biopsy site marker under ultrasound or other visualization modalities, may require that the patient undergo an additional procedure that places an additional device at the biopsy site to enable the surgeon to find the biopsy site in subsequent procedures. One known technique has been to place a breast lesion localization wire at the biopsy site. The localization wire is typically placed at the biopsy site via mammography and/or ultrasound.

Commonly assigned U.S. patent application Ser. No. 11/242,334 discloses a variety of markers. In some illustrations disclosed therein, expandable portions ‘hold’ a site marker in place within a biopsy cavity. That is, a site marker may include a bio-absorbable expandable portion with a marker, where the marker is visible under multiple modalities and the expandable portion will inhibit migration of the marker within the biopsy cavity. The expandable portions of these structures typically define a site marker diameter that is greater than the outer diameter of a cannula of a marker deployment device used to insert the site marker into the biopsy site.

In most cases, the marker deployment device is shipped with the site marker aligned with a distal opening of the cannula, and not with the site marker interposed within the cannula. This is because the site marker may take a heat set during sterilization. That is, when the site marker is sterilized within the cannula and then deployed into the biopsy site, the expandable portions of the site marker are unable to expand because the site marker has undergone the heat set during sterilization. During shipment, a wire may be used to hold the site marker in place along the distal opening.

The site marker sometimes separates from the distal opening during shipment, and hangs loose on the wire, away from the cannula distal opening. If the site marker separates from the distal opening, it is difficult to get the marker properly positioned and/or compressed for deployment of the site marker into a biopsy site. This is because it is difficult to re-align the site marker with the distal opening of cannula, as well as compress the marker into a desired configuration.

Accordingly, there is a need for deployment devices that retain the site marker in place along the distal opening of the cannula during shipment, and prior to deployment.

### BRIEF DESCRIPTION OF THE DRAWINGS

FIG. 1 is a partially sectioned side view of a site marker deployment system including a wire, a site marker, an inner cannula and an outer cannula;

FIG. 2 is a partially sectioned side view of an alternative illustration of the site marker and the wire, the inner cannula and the outer cannula;

FIG. 3 is a partially sectioned side view of the site marker in an expanded position, the wire, the inner cannula and an alternative illustration the outer cannula;

FIG. 3A is an enlarged plan view of a portion of the outer cannula illustrating a retention feature.

FIG. 4 is a partially sectioned side view of the site marker in a compressed position, the wire, the outer cannula and an alternative illustration the inner cannula; and

FIG. 5 is a partially sectioned side view of the marker deployment system in packaging.

### DETAILED DESCRIPTION

Referring now to the discussion that follows and also to the drawings, illustrative approaches to the disclosed systems and methods are shown in detail. Although the drawings represent some possible approaches, the drawings are not necessarily to scale and certain features may be exaggerated, removed, or partially sectioned to better illustrate and explain the present disclosure. Further, the descriptions set forth herein are not intended to be exhaustive or otherwise limit or restrict the claims to the precise forms and configurations shown in the drawings and disclosed in the following detailed description.

According to various exemplary illustrations described herein, a marker deployment system including a cannula, a site marker and at least one wire is provided. The cannula includes a tubular side-wall and a distal opening, and defines at least one aperture extending through the side-wall. The wire is selectively interposed within the cannula, and a distal wire end extends through the aperture. A portion of the wire is secured to an end portion of the site marker.

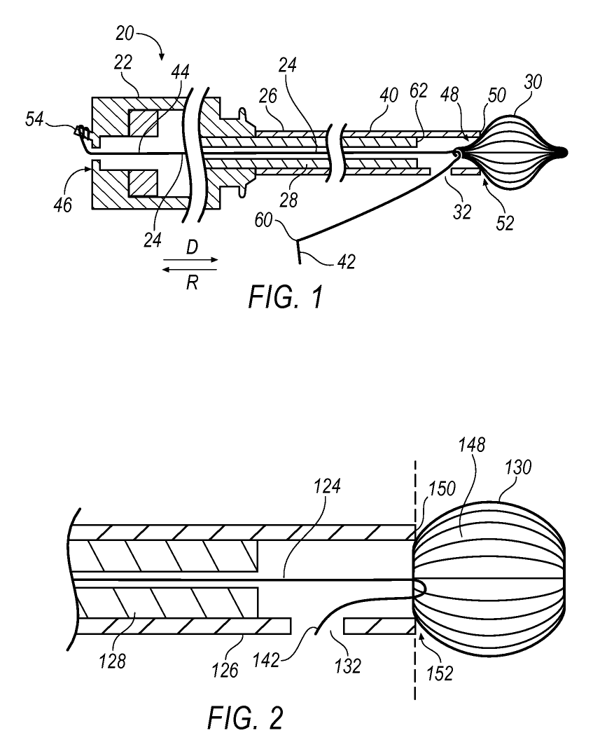

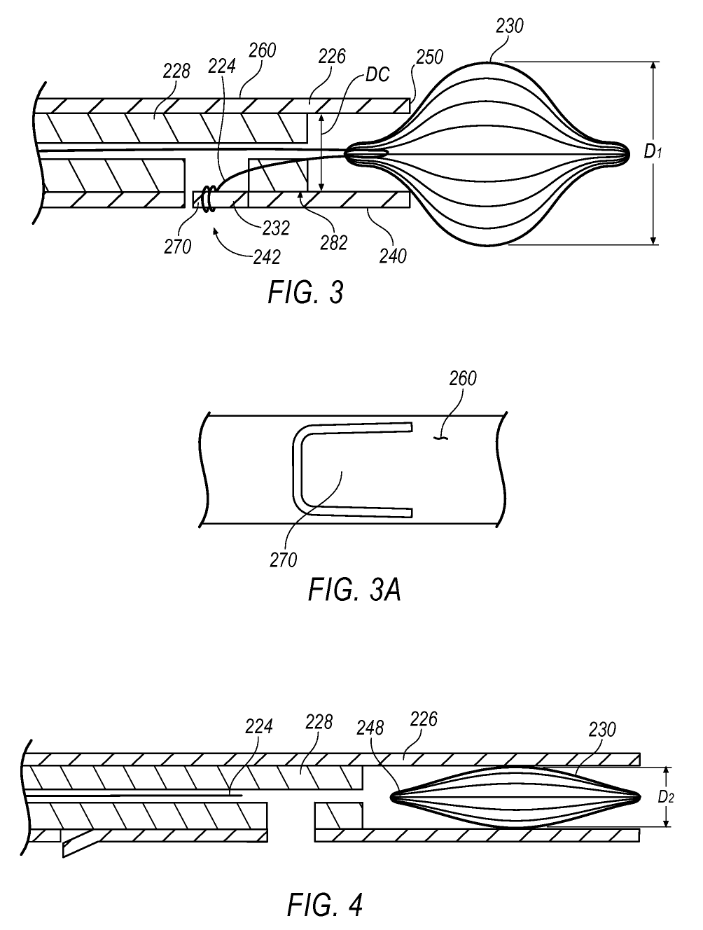

Turning now to the drawings and in particular to FIG. 1, an exemplary marker deployment system 20 is disclosed. In the illustration of FIG. 1, the marker deployment system 20 includes a marker deployment device 22, a wire 24, an outer cannula 26, an inner cannula 28 and a site marker 30. In the illustration as shown, the outer cannula 26 includes at least one aperture 32 that is formed along a portion of a tubular side-wall 40. The outer cannula 26 includes a distal opening 50, and the inner cannula 28 includes a distal opening 62. The distal opening 50 may be formed by a blunt edges, as shown, or may alternatively be chamfered or otherwised shaped. The inner cannula 28 is selectively interposed within the outer cannula 26. It should be noted that while FIG. 1 illustrates the aperture 32 extending through the side-wall 40 of the outer cannula 26, the aperture 32 may also extend from both of the inner cannula 28 and the outer cannula 26 as well. Indeed, as discussed in greater detail below, and as illustrated in FIG. 3, the aperture 232 may also extend through the side-wall 240 of the outer cannula 226 as well as through a side-wall 282 of the inner cannula 228. It is also noted that the aperture 32 is not limited to any particular size. Indeed, the aperture 32 only needs to be large enough to enable the wire 24 to extend therethrough, as will be explained below, in further detail. The inner and outer cannulas may be constructed from any suitable material. For example, the inner and outer cannulas may be constructed from a radiolucent material that would enable viewing under various imaging modalities.

The wire 24 is selectively interposed within the inner cannula 28 as well as the outer cannula 26, and includes a distal end 42 and a proximate end 44. The proximate end 44 of the wire 24 is secured to a proximate end 46 of the marker deployment system 20. In the illustration of FIG. 1, the proximate end 44 of the wire 24 is secured to a retention feature 54 that is located at the proximate end 46 of the marker deployment device 20. The wire 24 can be removed from the retention feature 54 prior to deployment of the site marker 30, as discussed in greater detail below.

A portion of the wire 24 is secured to an end portion 48 of the site marker 30. The distal end 42 of the wire 24 is then guided to the aperture 32 of the outer cannula 26, and the distal end 42 extends through the aperture 32. More specifically, the distal end 42 of the wire 24 is either aligned with or protrudes from the aperture 32.

The wire 24 is then pulled taut between the distal end 42 and the proximate end 44 of the outer cannula 26. That is, the wire 24 is pulled taut at the aperture 32, thereby tightening the portion of the wire 24 that is located within the outer cannula 26. Because the wire 24 is pulled tightly, the site marker 30 is thereby at least partially aligned with the distal opening 50. More specifically, the wire 24 is pulled through the aperture 32, thereby aligning the end portion 48 of the site marker 30 with a distal end 52 of the outer cannula 26. Typically, the wire 24 is pulled tight during assembly of the marker deployment system 20, prior to shipment. Because the wire 24 is pulled tightly during assembly, the site marker 30 can not separate from the distal opening 50 during shipment.

In the illustration as shown in FIG. 1, a portion of the site marker 30 is interposed within the outer cannula 26. However, in an alternative illustration as seen in FIG. 2, the end portion 148 of the site marker 130 may also be aligned with the distal opening 150 of the outer cannula 126, but not interposed within the distal end 152 of the outer cannula 126. In yet another embodiment, the site marker 130 may be substantially interposed within the outer cannula 126, as illustrated in FIG. 4.

In one exemplary illustration, the distal end 42 of the wire 24 is first pulled taut and extends past the aperture 32, as seen in FIG. 1. Thus, the distal end 42 of the wire 24 is located outside of the outer cannula 26, and protrudes from the aperture 32. In an alternative illustration, as seen in FIG. 2, the distal end 142 of the wire 124 only extends to and is aligned with the aperture 132. It should be noted that the distal end 42 of the wire 24 may be protruded past the aperture 32 of the outer cannula 26 as seen in FIG. 1, and then trimmed off such that the distal end 142 is aligned with the aperture 132 as seen in FIG. 2.

The wire 24 is constructed from any material that is sufficiently stiff to be held in place inside of the outer cannula 26, such as, but not limited to, a metal or a polymer that will substantially retain stiffness. The wire 24 may also include a locating feature for orienting the distal end 42 of the wire 24 at the aperture 32. For example, FIG. 1 illustrates the distal end 42 including a crimp 60 that is used to align the distal end 42 with the aperture 32. Although FIG. 1 illustrates the locating feature as the crimp 60, any feature that is able to locate the distal end 42 of the wire 24 to the aperture 32 along the side-wall 40 may be used as well.

In one exemplary illustration, as seen in FIG. 3, an outer surface 260 of the outer cannula 226 includes a retention feature 270 that is formed by a cut-out in the side wall of the outer cannula 226, as illustrated in FIG. 3A. In one embodiment (FIG. 3), the retention feature Ties substantially in the same plane as the side-wall of the outer cannula 226. In another embodiment, the retention feature 270 may be oriented to extend at least slightly outwardly from the outer surface 260. The distal end 242 of the wire 224 is secured to the retention feature 270. In the illustration as shown, the wire 224 is wrapped around the retention feature 270. Thus, because the distal end 242 of the wire 224 is secured to the retention feature 270, the wire 224 is held taut, and thereby holds the site marker 230 in place within the distal opening 250 of the outer cannula 226. FIG. 3 also illustrates the aperture 232 extending through the side-wall 240 of the outer cannula 226, as well as through a side-wall 282 of the inner cannula 228.

The site marker 230 is an expandable marker. That is, the site marker 230 is able to expand and compress. In the illustration as shown, the expendable marker 230 is displayed as an expendable filament type marker. However, it is understood that other types of expendable markers may also be used with the marker deployment device 20.

FIG. 3 illustrates the site marker 230 in the expanded position. FIG. 4 illustrates the site marker 230 in a compressed position. FIGS. 3-4 illustrate the site marker 230 including a first dimension D1 as well as a second dimension D2. The site marker 230 is expandable between the first dimension D1 and the second dimension D2. The first dimension D1 is illustrated in FIG. 3 and measures the diameter of the site marker 230. The first dimension D1 and the second dimension D2 are measured along a maximum diameter of the site marker 230.

Prior to deployment of the site marker 230, the wire 224 is retracted inside of the inner cannula 228. In the illustration of FIG. 1, the proximate end 44 of the wire 24 is removed from the retention feature 54. The wire 24 is then pulled in a direction D, towards the proximate end 46 of the marker deployment device 20. Although FIG. 1 illustrates the wire 24 being retracted inside of the inner cannula 28 by a user pulling the wire 24 manually, it should be noted that a variety of different methods utilizing the inner cannula 28 and the outer cannula 26 may be used to retract the wire 24 as well.

As seen in FIG. 4, when the wire 224 is retracted inside of the inner cannula 228, the portion of the wire 224 that was secured to the end portion 248 of the site marker 230 is removed from the end portion 248. In the illustration as shown, the wire 224 is retracted such that the wire 224 is wholly contained within the inner cannula 228. However, it should be noted that the wire 224 may be located within the outer cannula 226 when retracted as well.

When the wire 224 is retracted, the site marker 230 is urged inside the outer cannula 226. That is, the site marker compresses from the first dimension D1 to the second dimension D2, where the first dimension D1 is greater than the second dimension D2. The first dimension D1 is also greater than a diameter DC of the outer cannula 226, thus the site marker 230 is unable to be wholly interposed within the outer cannula 226 when the site marker 230 includes the first dimension D1. When the site marker 230 is compressed to the second dimension D2, the site marker 230 is able to fit within the outer cannula 226. In the illustration as shown in FIG. 4, the site marker 230 is wholly interposed within the outer cannula 226 when in the compressed position. However, in one embodiment, the inner cannula 28 is constructed such that passageway through which the wire 24 extends is sized to be sufficiently small to prevent the site marker 230 from being interposed within the passageway, even when the site marker 230 is compressed.

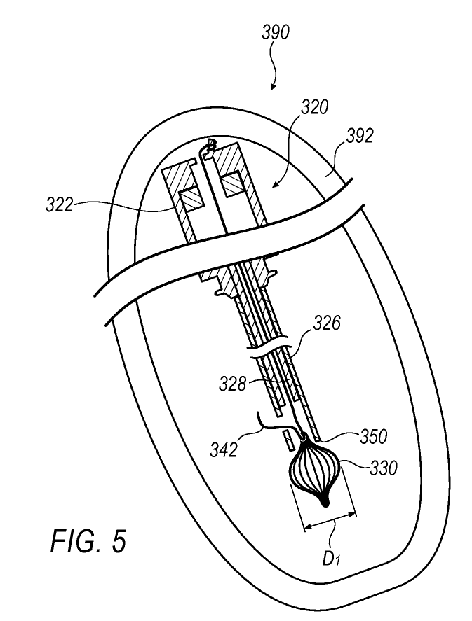

FIG. 5 illustrates the marker deployment system 320 that is included as part of a system 390 that is supplied in a sterilization configuration. The deployment device 322 may be sterilized prior to deployment of the site marker 330. In the illustration as shown, the wire 342 retains the site marker 330 at least partially within the outer cannula 326. Thus positioned, the site marker 330 is sterilized while in the expanded position. That is, the site maker includes the first dimension D1. Therefore, the site marker 330 is sterilized prior to being compressed into the second dimension D2.

The system 390 includes a packaging 392. In the illustration as shown, the site marker 330 is prevented from moving or shifting outside of the distal end 350 of the outer cannula 326. More importantly, the site marker 330 is retained in place until the deployment device 322 is removed from the packaging 392, and the wire 342 is retracted into the inner cannula 328. Collectively, the site marker 330, the deployment device 322, and the packaging 392 comprise the system 390.

In general, the site markers described herein may be made from biocompatible materials such as, but not limited to, titanium, stainless steel, and platinum. These materials have appropriate densities for radiographic imaging, appropriate surface characteristics for ultrasonic imaging, and appropriate magnetic characteristics for magnetic resonance imaging. The site markers are preferably made from titanium; however, it is understood that any suitable biocompatible material may be used. Portions of the site markers may be made from bioabsorbable materials. Commonly owned U.S. patent application Ser. Nos. 11/242,334, 10/964,087 and 11/561,919 disclose a variety of site markers that may be used in conjunction with the deployment device 322.

While the present disclosure has been particularly shown and described with reference to the foregoing preferred embodiments, it should be understood by those skilled in the art that various alternatives to the embodiments of the disclosure described herein may be employed in practicing the disclosure without departing from the spirit and scope of the disclosure as defined in the following claims. It is intended that the following claims define the scope of the disclosure embodiments within the scope of these claims and their equivalents be covered thereby. This description of the disclosure should be understood to include all novel and non-obvious combinations of elements described herein, and claims may be presented in this or a later application to any novel and non-obvious combination of these elements. The foregoing embodiment is illustrative, and no single feature or element is essential to all possible combinations that may be claimed in this or a later application.

What is claimed is:

1. A marker deployment system, comprising:
an outer cannula having a tubular side-wall and a distal opening, the outer cannula defining at least one aperture extending through the side-wall;
a site marker including an end portion; and
at least one wire including a distal wire end, the wire selectively interposed within the outer cannula, the distal wire end extending through the aperture;
wherein a portion of the wire is selectively connected to the end portion of the site marker;
wherein the wire is pulled taut at the aperture to at least partially align and retain the end portion of the site marker with the distal opening of the outer cannula.

2. The deployment system as recited in claim 1, wherein at least a portion of the site marker is interposed within the outer cannula when the wire is pulled taut.

3. The deployment system as recited in claim 1, wherein the distal wire end protrudes through the aperture.

4. The deployment system as recited in claim 1, wherein the outer cannula includes a retention feature formed from a section of the side wall of the outer cannula and extending outwardly from an outer surface of the outer cannula, the distal end of the wire being secured to the retention feature.

5. The deployment system as recited in claim 1, wherein the wire is constructed from one of a metal and a polymer.

6. The deployment system as recited in claim 1, wherein the distal end of the wire includes a locating feature.

7. The deployment system as recited in claim 6, wherein the locating feature is a crimped end.

8. The deployment system as recited in claim 1, wherein the site marker includes a first dimension and a second dimension, the second dimension being greater than the first dimension and greater than a diameter of the distal opening in the outer cannula, and the site marker being expandable between the first dimension and the second dimension.

9. The deployment system as recited in claim 1, further comprising an inner cannula interposed within the outer cannula, wherein the inner cannula is selectively slidable within the outer cannula and further defines a passageway therethrough that terminates in an open distal end, wherein the wire may be interposed within the passageway of the inner cannula.

10. The deployment system as recited in claim 1, further comprising an inner cannula having an inner cannula tubular side-wall and an inner cannula distal opening, the inner cannula further defining the aperture extending through the inner cannula side-wall.

11. A marker deployment system, comprising:
an outer cannula having an outer cannula tubular side-wall and an outer cannula distal opening, the outer cannula defining at least one aperture extending through the outer cannula side-wall;
an inner cannula selectively interposed within the outer cannula;
a site marker including an end portion; and
at least one wire including a distal wire end, the wire selectively interposed within the inner cannula and the outer cannula, the distal wire end extending through the aperture;
wherein a portion of the wire is selectively secured to the end portion of the site marker;
wherein the wire is pulled taut at the aperture to at least partially align the end portion of the site marker with the outer cannula distal opening.

12. The deployment system as recited in claim 11, wherein at least a portion of the site marker is interposed within the outer cannula when the wire is pulled taut.

13. The deployment system as recited in claim 11, wherein the distal wire end protrudes through the aperture.

14. The deployment system as recited in claim 11, wherein the outer cannula includes a retention feature formed from a section of the side wall of the outer cannula, wherein the retention feature extends outwardly from an outer surface of the outer cannula, the distal end of the wire being secured to the retention feature.

15. A method of assembling a marker deployment system, comprising:
interposing at least one wire within an outer cannula, the cannula having a tubular side-wall and a distal opening, the outer cannula defining at least one aperture extending through the side-wall, the wire having a distal end;
selectively securing a portion of the wire to an end portion of the site marker;
extending the distal end of the wire through the aperture; and
pulling the wire taut to at least partially align the end portion of the site marker with the distal opening of the cannula.

16. The method of claim 15, further comprising interposing an inner cannula having a passageway therethrough within the outer cannula and interposing at least one wire through the passageway of the inner cannula.

17. The method of claim 16, further comprising looping the wire through a portion of the site marker.

18. The method of claim 15, further comprising the step of orienting the distal end of the wire at the aperture of the outer cannula with a locating feature, the locating feature included at the distal end of the wire.

19. The method of claim 18, further comprising the step of protruding the distal end of the wire through the aperture.

20. The method of claim 19, further comprising the step of interposing at least a portion of the site marker within the outer cannula by pulling the wire.

21. The method of claim 20, further comprising the step of securing the distal end of the wire to a retention feature formed in side-wall of the outer cannula.

22. The method of claim 21, further comprising the step of trimming off a portion of the wire that protrudes past the aperture.
